(19) United States
(12) Patent Application Publication
(10) Pub. No.: US 2025/0349439 A1
(43) Pub. Date: Nov. 13, 2025

(54) PROGNOSTICATING RISK OF A CLINICAL OUTCOME

(71) Applicant: DIGISTAIN INC., Wilmington, DE (US)

(72) Inventor: Hemmel AMRANIA, Harrow Middlesex (GB)

(21) Appl. No.: 19/277,071

(22) Filed: Jul. 22, 2025

Related U.S. Application Data
(63) Continuation of application No. PCT/GB2024/050054, filed on Jan. 10, 2024.

(51) Int. Cl. G16H 50/30; G16H 10/40; G16H 10/60

(52) U.S. Cl. CPC G16H 50/30; G16H 10/40; G16H 10/60

(57) ABSTRACT

A method for prognosticating risk of a clinical outcome for a patient, the method comprising: receiving clinical data relating to the patient; generating a histological index based on infrared absorption data gathered from a sample of the patient; and generating a prognosticated risk score based on the histological index and the clinical data. The method may be partially or fully automated.

### CROSS-REFERENCE TO RELATED APPLICATION(S)

This patent document is a 371 National Phase Application of PCT Application No. PCT/GB2024/050054, entitled “PROGNOSTICATING RISK OF A CLINICAL OUTCOME,” filed on Jan. 10, 2024, which claims priority to and benefits of GB Application No. 2301020.0, filed Jan. 24, 2023, now issued GB Patent No. 2626541, issued Jul. 31, 2024 entitled “PROGNOSTICATING RISK OF A CLINICAL OUTCOME”. The entire content of the before-mentioned patent application is incorporated by reference as part of the disclosure of this document.

### TECHNICAL FIELD

The patent document relates to methods, computer programs and apparatus for prognosticating risk of a clinical outcome based on a tissue sample of a patient.

### BACKGROUND

In the field of medical analysis, there are several known methods estimating the risk of certain clinical outcomes occurring due to various diseases and conditions, such as cancers and other tumours for example. The estimated risk may be used to support decision making for subsequent therapy.

However, the available genomic tests each vary in different ways. Each has been designed to evaluate the expression of a different gene set and each has been clinically validated in different diagnostic settings. Hence, there is a relatively low concordance between them. Further, despite their clinical utility, their use in the management of cancer remains relatively moderate, even in better resourced healthcare systems.

Cost and reimbursement issues, as well as turnaround time between tissue acquisition and tissue preparation for actual testing have been reported as barriers to performing genomic testing of breast cancers in the community setting, for example. Any delays are important because there is significant inverse association between the initiation of adjuvant chemotherapy and survival in breast cancer. Also, from a patient perspective, any waiting time for test results and decisions regarding treatment only adds to anxiety and stress, critically underscoring the need for more rapid testing.

Further, known methods involving genomic risk profiling face challenges that arise due to unavoidable variabilities in tissue processing including the size of a tumour, the impact of slow penetration of formalin and variability in fixation time and RNA lability.

It is an object to provide a risk prognostication tool that can be implemented cost-effectively, with improved accuracy or with more rapid results.

### SUMMARY

According to one aspect, the invention provides a method for prognosticating risk of a clinical outcome for a patient, the method comprising:
receiving clinical data relating to the patient;
generating a histological index based on infrared absorption data gathered from a sample of the patient; and
generating a prognosticated risk score based on the histological index and the clinical data.

The method may be partially or fully automated.

In some embodiments, generating the prognosticated risk score may comprise incorporating the histological index and the clinical data in a model.

In some embodiments, the model may be a multivariate logistic model.

Some embodiments comprise calculating a hazard ratio for each of the histological index and the clinical data. The clinical data may comprise data of different types, in which case a hazard ratio may be determined for each type of data. The hazard ratio may be determined using a logistic regression model, such as the Cox regression model.

In some embodiments, generating the prognosticated risk score further may comprise incorporating a hazard ratio into the model, the hazard ratio calculated using a logistic regression model.

In some embodiments, the method may further comprise:
using the prognosticated risk score to stratify the patient into one of at least two risk classifications with respect to the clinical outcome.

In some embodiments, the method may further comprise:
determining one or more clinicopathological factor values based on the clinical data,
wherein the or each clinicopathological factor value is used to generate the prognosticated risk score.

In some embodiments, the clinical outcome may comprise:
death;
disease-specific death;
recurrence; or
complete pathological response.

In some embodiments, the patient may be a patient previously diagnosed with a cancer.

In some embodiments, the cancer may be one of breast cancer, colon cancer, prostate cancer or renal cancer.

In some embodiments, the patient may have a known medical condition, wherein the clinical data is an attribute of the patient associated with prognosis for the known health condition.

In some embodiments, the clinical data may comprise one or more of:
age when diagnosed with cancer;
menopausal status;
tumour size;
tumour grade; and/or
lymph node status.

In some embodiments, age when diagnosed with cancer and/or tumour size may be modelled as continuous variables. Similarly, menopausal status, tumour grade and/or lymph node status may be modelled as categorical variables.

In some embodiments, generating the histological index based on infrared absorption data gathered from the sample may comprise:
gathering infrared absorption data from the sample at selected wavelengths;
determining, from the infrared absorption data, a first measure of the amount of energy or power absorbed attributable to an amide moiety and a second measure of the amount of energy or power absorbed attributable to a phosphate moiety; and
determining a ratio of the first measure and the second measure to establish the histological index.

In some embodiments, the selected wavelengths may lie in the ranges 6.0±0.5 microns, 6.47±0.50 microns, 8.13±0.44 microns, 9.3±0.7 microns.

In some embodiments, the histological index may comprise a numeric value obtained by dividing the first measure by the second measure.

In some embodiments, the histological index, PA, may be derived according to the expression PA=[|M(λ3)−(λ4)|]/[|(λ1)−(λ2)|] where: (λn) is a measure of the absorbed energy or power at λn; λ1 is a wavelength corresponding to a peak absorption value attributable to an amide moiety; λ2 is a wavelength corresponding to a baseline absorption value attributable to an amide moiety; λ3 is a wavelength corresponding to a peak absorption value attributable to a phosphate moiety; λ4 is a wavelength corresponding to a baseline absorption value attributable to a phosphate moiety.

In some embodiments, the histological index, PA, may be derived according to the expression PA=[X3 M(λ3)−X4 M(λ4)]/[X1 M(λ1)−X2 M(λ2)] where: M(λn) is a measure of the absorbed energy or power at λn; λ1 is a wavelength corresponding to a peak absorption value attributable to an amide moiety; λ2 is a wavelength corresponding to a baseline absorption value attributable to an amide moiety; λ3 is a wavelength corresponding to a peak absorption value attributable to a phosphate moiety; λ4 is a wavelength corresponding to a baseline absorption value attributable to a phosphate moiety; and X1 to X4 are numerical factors ≥1 which are set to values sufficient to ensure that the measure M for a peak absorption values λ3 and λ1 is always greater than the measure M for the corresponding baseline absorption values λ4 and λ2 for all measurements. X1 to X4 may all be different values or may include two or more of the same value.

According to a further aspect, the invention provides a computer program comprising computer code configured to perform a method according to the aspect referred to above, including any of its embodiments.

According to another aspect there is provided a non-transitory computer-readable storage medium comprising one or more computer programs for execution by one or more processors of a device, the one or more programs including instructions which, when executed by the one or more processors, cause the device to perform any method disclosed herein.

According to a further aspect, the invention provides a device for prognosticating risk of a clinical outcome for a patient comprising:
one or more processors;
a memory comprising one or more computer programs for execution by the one or more processors of a device, the one or more programs including instructions which, when executed by the one or more processors, cause the device to perform any method disclosed herein.

According to another aspect, the invention provides a method for prognosticating risk of a clinical outcome based on a sample of a patient, the method comprising:
receiving clinical data relating to the patient;
gathering infrared absorption data from the sample at selected wavelengths;
generating a histological index based on the gathered infrared absorption data; and
generating a prognosticated risk score based on the histological index and the clinical data.

In some embodiments, the infrared absorption data may be gathered using an interferometer, Raman spectroscopy, spectral imager, spectral detector and/or a wavelength-tuneable light source.

In some embodiments, the sample may be a tissue sample.

In some embodiments, the sample may be from 1 μm to 10 μm thick, preferably about 4 μm thick.

In some embodiments, the sample may comprise human or animal tissue.

In some embodiments, the sample may comprise breast tissue.

In some embodiments, the sample may comprise frozen tissue, formalin fixed tissue or liquid serum.

In some embodiments, the obtained infrared absorption data may relate to a single spatial position on the sample.

In some embodiments, the obtained infrared absorption data may relate to a spot size of greater than 100 microns. For example, a cross sectional dimension of the spot may be greater than 100 microns, 200 microns or 500 microns. A cross sectional dimension of the spot may be less than 1200 microns, 1000 microns or 800 microns.

According to further aspect, the invention provides an apparatus for prognosticating risk of a clinical outcome based on a sample of a patient, comprising:
a detector configured to obtain infrared absorption data from a tissue at selected wavelengths; and
a processing module configured to process said infrared absorption data and receive clinical data related to the patient,
wherein the apparatus is configured to carry out the method of the aspect referred to above, including any of its embodiments.

In some embodiments, the detector may be comprised by an interferometer.

### BRIEF DESCRIPTION OF THE DRAWINGS

Embodiments of the present invention will now be described by way of example and with reference to the accompanying drawings in which:

FIG. 1 shows a schematic diagram of an apparatus for acquiring infrared absorption data from a sample;

FIG. 2 shows a schematic example of an infrared absorption spectrum obtained from a sample;

FIG. 3 is a flow diagram illustrating steps of a data processing technique suitable for generating a histological index;

FIG. 4 shows a graph illustrating the correlation between a known cancer grading method and a histological index;

FIG. 5 shows a flow diagram illustrating steps of a method for prognosticating risk of a clinical outcome based on a histological index;

FIG. 6 shows a flow diagram illustrating steps of another method for prognosticating risk of a clinical outcome based on a tissue sample;

FIG. 7 shows a box plot of histological index distribution against pleomorphism scores;

FIG. 8 shows graph illustrating histological index accuracy (AUC under ROC curves) at 5-years for predicting disease free survival in HR+/HER2−/LN+≤3 patients;

FIG. 9 shows graph illustrating histological index accuracy (AUC under ROC curves) at 10-years for predicting disease free survival in HR+/HER2−/LN+≤3 patients;

FIG. 10 shows a Kaplan-Meier curve for disease free survival based on histological index classification for high and low risk in HR+/HER2−/LN+≤3 patients, indicating distribution of events at years of follow-up;

FIG. 11 shows graph illustrating histological index accuracy similar to FIG. 8, except for predicting recurrence;

FIG. 12 shows graph illustrating histological index accuracy similar to FIG. 9, except for predicting recurrence;

FIG. 13 shows a Kaplan-Meier curve similar to FIG. 10, except for predicting recurrence;

FIG. 14 shows graph illustrating histological index accuracy similar to FIG. 8, except for predicting overall survival;

FIG. 15 shows graph illustrating histological index accuracy similar to FIG. 9, except for predicting overall survival;

FIG. 16 shows a Kaplan-Meier curve similar to FIG. 10, except for predicting overall survival;

FIG. 17 shows graph illustrating histological index accuracy similar to FIG. 8, except for HR+/HER2−/LN− patients;

FIG. 18 shows graph illustrating histological index accuracy similar to FIG. 9, except for HR+/HER2−/LN− patients;

FIG. 19 shows a Kaplan-Meier curve similar to FIG. 10, except for HR+/HER2−/LN− patients;

FIG. 20 shows graph illustrating histological index accuracy similar to FIG. 11, except for HR+/HER2−/LN− patients;

FIG. 21 shows graph illustrating histological index accuracy similar to FIG. 12, except for HR+/HER2−/LN− patients;

FIG. 22 shows a Kaplan-Meier curve similar to FIG. 13, except for HR+/HER2−/LN− patients;

FIG. 23 shows graph illustrating histological index accuracy similar to FIG. 14, except for HR+/HER2−/LN− patients;

FIG. 24 shows graph illustrating histological index accuracy similar to FIG. 15, except for HR+/HER2−/LN− patients;

FIG. 25 shows a Kaplan-Meier curve similar to FIG. 16, except for HR+/HER2−/LN− patients;

FIG. 26 shows graph illustrating histological index accuracy similar to FIG. 8, except for premenopausal patients (age ≤45 years);

FIG. 27 shows graph illustrating histological index accuracy similar to FIG. 9, except for premenopausal patients (age ≤45 years);

FIG. 28 shows a Kaplan-Meier curve similar to FIG. 10, except for premenopausal patients (age ≤45 years);

FIG. 29 shows graph illustrating histological index accuracy similar to FIG. 11, except for premenopausal patients (age ≤45 years);

FIG. 30 shows graph illustrating histological index accuracy similar to FIG. 12, except for 3 premenopausal patients (age ≤45 years);

FIG. 31 shows a Kaplan-Meier similar to FIG. 13, except for premenopausal patients (age ≤45 years);

FIG. 32 shows graph illustrating histological index accuracy similar to FIG. 14, except for 3 premenopausal patients (age ≤45 years);

FIG. 33 shows graph illustrating histological index accuracy similar to FIG. 15, except for premenopausal patients (age ≤45 years);

FIG. 34 shows a Kaplan-Meier curve similar to FIG. 16, except for premenopausal patients (age ≤45 years);

FIG. 35 shows graph illustrating histological index accuracy similar to FIG. 8, except for postmenopausal patients (age ≥60 years);

FIG. 36 shows graph illustrating histological index accuracy similar to FIG. 9, except for postmenopausal patients (age ≥60 years);

FIG. 37 shows a Kaplan-Meier curve similar to FIG. 10, except for postmenopausal patients (age ≥60 years);

FIG. 38 shows graph illustrating histological index accuracy similar to FIG. 11, except for postmenopausal patients (age ≥60 years);

FIG. 39 shows graph illustrating histological index accuracy similar to FIG. 12, except for postmenopausal patients (age ≥60 years);

FIG. 40 shows a Kaplan-Meier curve similar to FIG. 13, except for postmenopausal patients (age ≥60 years;

FIG. 41 shows graph illustrating histological index accuracy similar to FIG. 14, except for postmenopausal patients (age ≥60 years);

FIG. 42 shows graph illustrating histological index accuracy similar to FIG. 15, except for postmenopausal patients (age ≥60 years); and

FIG. 43 shows a Kaplan-Meier curve similar to FIG. 16, except for postmenopausal patients (age ≥60 years).

### DETAILED DESCRIPTION

Most biological molecules have vibrational modes with wavelengths which lie in the mid-infrared spectral range between 3 μm and about 16 μm. The positions, width and strength of the vibrational modes vary with composition and structure of the molecule. Identification of vibrational modes of major biological molecules, such as proteins, lipids and nucleic acids, can be determined by Fourier transform infrared spectroscopy. Infrared radiation directed at a biological sample (e.g., a tissue sample) is variously absorbed or transmitted depending on the biological material present (i.e., compounds and functional groups present in the sample) as well as the concentration and distribution of the material in the sample. The sample's infrared spectrum exhibits characterising spectral features such as absorption bands of characteristic shape and size at characteristic frequencies. These characterising spectral features act as “fingerprints” by which to identify uniquely the presence of a particular functional group; moreover, the presence of a certain functional group is indicative of a certain biological molecule.

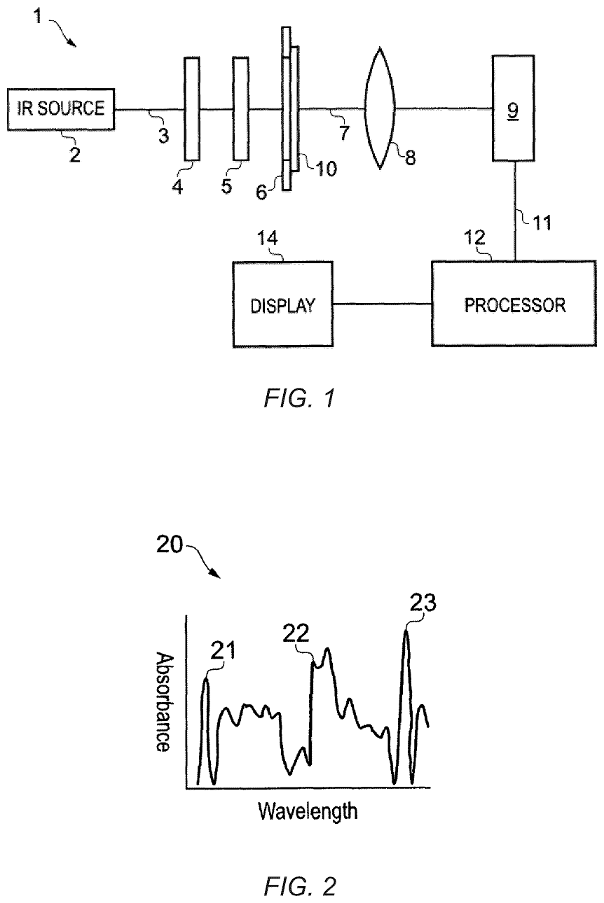

FIG. 1 shows an apparatus 1 according to an aspect. The apparatus 1 is suitable for acquiring infrared absorption data from a sample 10 ex vivo. An infrared source 2 provides an output of infrared radiation 3 which can pass through a shutter 4 (when open) and a filter 5 to reach the sample 10, which is mounted on a sample stage 6. The sample 10 either absorbs the infrared radiation or transmits the infrared radiation according to the local structure and/or composition of the sample 10 and the frequency/wavelength of the radiation. Transmitted infrared radiation 7, which passes through the sample 10, is focused by a focusing element 8 onto a suitable detector 9.

The infrared source 2 can be of any suitable type, and preferably one capable of efficiently producing infrared output 3 with wavelengths in the mid-infrared range, particularly a range of approximately 5 to 9 microns.

The shutter 4 may comprise any suitable arrangement capable of interrupting the infrared output 3 to prevent it reaching the sample 10.

The filter 5 may be any suitable device for enabling only selected wavebands of infrared output 3 to reach the sample 10. For example, the filter 5 may be a filter wheel, comprising multiple separate filter elements which can be moved into the beam line, or could be a tuneable filter. More generally, the filter 5 may be a controllable filter for enabling selection of a wavelength or narrow range of wavelengths of infrared radiation to reach the sample 10. In this way, separate infrared absorption measurements may be made on the sample at specific wavelengths or in specific wavebands at different times.

Transmitted infrared radiation 7 not absorbed at a particular spatial position on the sample 10 may be focused onto the detector 9 using any suitable focusing element 8, which may be a lens or multiple lenses.

The detector 9 may be any suitable device such as an interferometer, a bolometric camera Raman Spectrometer or any detector sensitive to infrared radiation. In particular, the detector 9 may be a Fourier Transform Infrared Spectroscopy Interferometer.

If, for example, a Raman Spectrometer was to be used in place of a Fourier Transform Infrared Spectroscopy Interferometer, suitable adjustments of the methods disclosed herein would be readily understood by a person skilled in the art. Raman Spectroscopy may provide a particularly suitable alternative to Fourier Transform Infrared Spectroscopy by virtue of being able to obtain similar data from a sample. Raman Spectroscopy also facilitates data collection from a spot, potentially with greater specificity than may be achieved using Fourier Transform Infrared Spectroscopy.

In an alternative arrangement, the broadband infrared source 2, shutter 4 and filter wheel 5 might be replaced with a switchable and/or tuneable infrared light source (not shown) capable of generating infrared output beams 3 at different wavelengths, such as an optical parameter generator or optical parametric oscillator, or one or more quantum cascade lasers.

In some embodiments, the infrared source 2 and detector 9 are configured to irradiate the entire sample 10 simultaneously, or substantial parts thereof, and to capture transmitted infrared radiation 7 for a large spatial area of the sample 10 in a single exposure. In other embodiments, the apparatus 1 may sample only small parts of the sample 10 at a time and use a position controllable sample stage 6 to sequentially measure absorption in different parts of the sample (e.g., in multiple exposures).

An output 11 from the detector 9 is passed through a suitable processing device or processor 12 to perform analysis functions described hereinafter. The processor 12 may be coupled to a suitable display 14. The display 14 may also serve as a user input device.

Infrared absorption measurements may be taken by comparing detector measurements taken with the sample 10 in position on the sample stage 6 and those taken with the sample 10 removed from the sample stage 6.

Absorption measurements at each of four wavelengths λ1, λ2, λ3, λ4, may be taken in any suitable manner according to the infrared absorption analysis apparatus used. Each absorption measurement Man may be taken using four measurements:
(i) a sample image S taken with the sample 10 loaded in the apparatus 1, on the sample stage 6, and the shutter 4 open/removed;
(ii) an environment signal Es taken with the sample 10 loaded in the apparatus 1 and the shutter 4 closed/in place;
(ii) a background image B taken with the sample 10 out of the apparatus 1 and the shutter 4 open/removed; and
(iv) a background environment Eb measurement taken with the sample 10 out and the shutter 4 closed/in place.

The absorption measurements M_{λn}, each corresponding to an absorbed power or energy, are derived according to: M_{λn}=(S−Es)/(B−Eb). Preferably, each absorption measurement Man is taken a number of times (e.g., N times) and a mean value of Man is calculated. The value of N may be selected according to the acquisition time set for the apparatus 1.

FIG. 2 shows schematically an exemplary infrared absorption spectrum 20 obtained from a sample, such as the sample 10. The infrared absorption spectrum 20 may be derived as a function of an infrared transmittance spectrum received by the detector 9 using techniques known in the art. Peaks 21, 22, 23 each correspond to spectral features that are associated with a particular functional group. These groups may typically include functional groups such as those listed in the tables below.

TABLE 1
Spectral features associated with certain functional groups.

| Functional | | | Phosphodiester | Phosphodiester |
|---|---|---|---|---|
| Group | νₛPO₄⁻ | νₛPO₄⁻ | PO₂⁻ | PO₂⁻ |
| Associated | Phosphorylated | oRNA | nucleic acid | nucleic acid |
| biological | proteins and | | and lipids | and lipids |
| molecule | nucleic acid | | | |
| Wavenumber | 964 | 995 | 1240 | 1080 |
| (cm⁻¹) | | | | |
| Wavelength | 10.37 | 10.05 | 8.06 | 9.26 |
| (μm) | | | | |
| Frequency | 28.9 | 29.9 | 37.2 | 32.4 |
| (THz) | | | | |

TABLE 2
Spectral features associated with further functional groups.

| | | | | Methylene | |
|---|---|---|---|---|---|
| | | | | CH₂ and | |
| Functional | Amide II | Carbonyl | Amide I | Methyl | OH and |
| Group | CO—NH | CO | CO—NH | CH₃ | NH |
| Associated | proteins | Lipids | proteins | proteins | Proteins |
| biological | | | | and lipids | and |
| molecule | | | | | polysac- |
| | | | | | charides |
| Wavenumber | 1545 | 1740 | 1650 | 2850-2960 | 3300 |
| (cm⁻¹) | | | | | |
| Wavelength | 6.47 | 5.75 | 6.06 | 3.51-3.38 | 3.03 |
| (μm) | | | | | |
| Frequency | 46.3 | 52.2 | 49.5 | 85.4-88.7 | 99.0 |
| (THz) | | | | | |

Determining a first measure M_{A} of an amount of power or energy absorbed which is attributable to an amide functional group/amide moiety and a second measure M_{P} of an amount of power or energy absorbed which is attributable to a phosphate functional group/phosphate moiety, and computing a ratio of the first measure and the second measure provides a quantitative measure which can serve as a useful histological index which is a measure reflective of a measure reflective of aneuploidy (i.e., the condition of having an abnormal number of chromosomes in a haploid set) and hence a reliable prognostic marker, at least for invasive breast cancer and feasibly for other types of cancer such as colon cancer and prostate cancer.

More generally, the ratio M_{P}/M_{A} that defines this histological index provides a useful indicator of the tissue structure or structures present in the sample that are highly relevant to further clinical assessment. The ratio may therefore be extremely useful in automation of a first stage of a screening program for individuals at risk of certain types of cancer and may be reliably indicative of a clinical prognosis. The tissue type may be breast tissue or other types of tissue and the types of cancer may include breast cancer or other types of cancer, such as colon cancer and prostate cancer.

The first measure M_{A} may be a measure of infrared absorption (preferably absorbed power) attributable to an amide functional group taken in the range 5.5 to 6.5 microns wavelength and the second measure M_{P} may be a measure of infrared absorption attributable to a phosphate functional group taken in the range 7.6 to 8.6 microns or 8.6 to 10.0 microns. The measurements MA and MP may therefore comprise, correspond to, or approximate, respectively, an area under the amide absorption peak and an area under the phosphate absorption peak. The first measure M_{A} may comprise a computed area under the absorption peak found at 6.0 microns wavelength and the second measure M_{P} may comprise a computed area under the absorption peak found at 8.13 microns wavelength or a computed area under the absorption peak found at 9.3 microns wavelength.

The first measure M_{A} may comprise a measure of infrared absorption attributable to an amide functional group taken in the range 6.0±0.5 microns wavelength or 6.47±0.5 microns and the second measure M_{P} may comprise a measure of infrared absorption attributable to a phosphate functional group taken in the range 8.13±0.44 microns or 9.3±0.7 microns. The ranges given above preferably represent the full width at half maximum of the peak. The infrared absorption by the phosphate functional group/phosphodiester concentration is preferably measured at 8.13 microns because, although the peak at 9.3 microns gives better contrast with respect to a baseline in some cases, the signal-to-noise ratio at this wavelength may be limited due to low emissivity of some infra-red sources. However, improvements in signal-to-noise ratio may shift this preference.

The first measure M_{A} may comprise a difference between an absorption measurement taken at an absorption peak for an amide functional group and an absorption measurement taken at a baseline of the absorption peak for the amide functional group. The second measure M_{P} may comprise a difference between an absorption measurement taken at an absorption peak for a phosphate functional group and an absorption measurement taken at a baseline of the absorption peak for the phosphate functional group.

Fore example, the difference between an absorption measurement taken at an absorption peak for an amide functional group and an absorption measurement taken at a baseline of the absorption peak for the amide functional group may be established by: a) measuring an absorption peak at λ1=6.0 microns, and b) measuring an absorption baseline at λ2=6.47 microns.

Similarly, the difference between an absorption measurement taken at an absorption peak for a phosphate functional group and an absorption measurement taken at a baseline of the absorption peak for the phosphate functional group may be established by: a) measuring an absorption peak at λ3=8.13 microns, and b) measuring an absorption baseline at λ4=8.57 microns.

The difference between measurements may be established by using a scaling factor as will be described below.

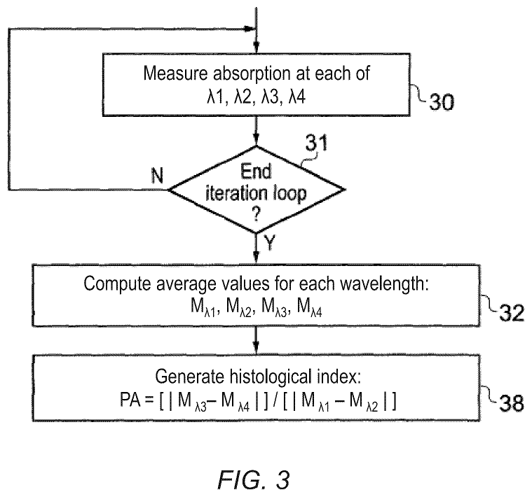

FIG. 3 shows a flow chart of a method suitable for generating a histological index based on a tissue sample. The method involves data processing which may be performed within the processor 12 of FIG. 1.

First, the absorption is measured at each of λ1, λ2, λ3, λ4 (step 30) for a single spatial position in the tissue sample. The selected wavelengths may, for example, lie in the ranges 6.0±0.5 microns, 6.47±0.50 microns, 8.13±0.44 microns, 9.3±0.7 microns, respectively.

This is repeated a number, N, times (step 31) to generate N wavelength measurements for the single spatial position. N may be varied according to system parameters including acquisition time, to optimise performance. An average value (which may be the mean) is computed (step 32) to create a dataset Man comprising an average value of absorption at each wavelength.

A value corresponding to the histological index, PA, is then generated from the four average value of absorption M_{λ1}, M_{λ2}, M_{λ3}, M_{λ4} (step 38). The histological index may be determined as a ratio of the first measure and the second measure. More particularly, the histological index may comprise a numeric value obtained by dividing the first measure by the second measure. In other words, the histological index may be derived according to the expression shown in step 38:

$$PA = [ \vert {M}_{λ 3} - {M}_{λ 4} \vert ] / [ \vert {M}_{λ 1} - {M}_{λ 2} \vert ]$$

More generally, M_{λn} is a measure of the absorbed energy or power at λn; λ1 is a wavelength corresponding to a peak absorption value attributable to an amide moiety; λ2 is a wavelength corresponding to a baseline absorption value attributable to an amide moiety; λ3 is a wavelength corresponding to a peak absorption value attributable to a phosphate moiety; and λ4 is a wavelength corresponding to a baseline absorption value attributable to a phosphate moiety.

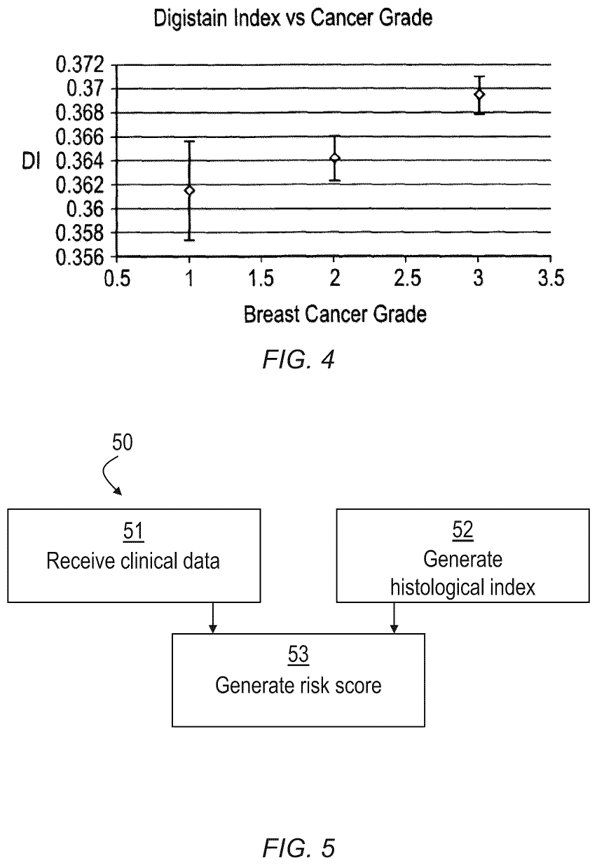

FIG. 4 shows a graph illustrating a correlation between conventional breast cancer grading (horizontal axis) using staining methods and the histological index (or Digistain Index, DI) described above (vertical axis). In other words, the graph demonstrates the utility of a histological index generated as described above for indicating the severity of a cancer, particularly a breast cancer.

However, although the histological index has been found by the inventor to be a very useful tool, it is not possible to gain a more general appreciation of a patient's prognosis from a histological index alone. The reason for this is that it does not take into account other potentially important factors such as particular attributes of a patient that may have a significant bearing on the clinical outcome for the patient.

FIG. 5 shows a flow diagram representing a method 50 suitable for prognosticating risk of a clinical outcome based on a sample of a patient.

In this example, the patient is a patient who has been previously diagnosed with breast cancer. However, it is to be understood that methods may be applicable to patients diagnosed with other types of cancer, such as colon cancer or prostate cancer. In a general sense, the patient is a subject with a known medical condition or past medical condition associated with the sample. For example, the patient may be known to have or may have had a cancer or other tumour.

The clinical outcome, the risk of which is being prognosticated, may be death, disease-specific death or recurrence of cancer.

The method 50 comprises initial steps of receiving clinical data relating to the patient (step 51). The clinical data is typically an attribute of the patient diagnosed with the medical condition that is of a type that has a statistically significant bearing on the clinical outcome for the patient. For example, the clinical data may comprise one or more of: age when diagnosed with cancer; menopausal status; tumour size; tumour grade; and/or lymph node status (i.e., the number of lymph nodes in the underarm area, also referred to as axillary lymph nodes, contain cancer). It will be appreciated that different clinical data may be selected dependent on the condition.

The method also includes generating a histological index based on infrared absorption data gathered from the sample (step 52). Step 52 may be carried out using the apparatus 1, shown in FIG. 1, according to the method illustrated in FIG. 3.

Steps 51 and 52 may be carried out simultaneously or sequentially in any order.

The method 50 then comprises the further step of generating a prognosticated risk score based on the histological index and the clinical data (step 53).

Step 53 may comprise determining one or more clinicopathological factor values (CFVs) based on the clinical data, wherein the, or each, CFV is used to generate the prognosticated risk score.

The CFVs may be modelled as continuous or categorical values. For example, clinical data relating to age when diagnosed with cancer and/or tumour size may be modelled as continuous variables. If necessary, some form of scaling, normalisation or other adjustment may be applied to the raw data to account for factors such as skewness. For example, a natural logarithm of the raw data may be used.

Meanwhile, clinical data relating to menopausal status, tumour grade and/or lymph node status may be modelled as categorical variables. For example, tumours may be graded as grade 1, 2 or 3 depending on severity and the grade may be used as the CFV which is then applied as a variable within the model. Similarly, lymph node status may be assigned as “1” for negative (i.e., no lymph nodes containing cancer) or “2” for any of 1-3 positive lymph nodes/lymph nodes containing cancer.

By incorporating the clinical data and objective information from spectral measurements into the prognosticated risk score in this way, the invention is less susceptible to subjectivity issues that known prognostic tools based on clinicopathological features (e.g., NPI or PREDICT) suffer with.

Step 53 may comprise incorporating the histological index and the clinical data in a model, optionally a multivariate logistic model as discussed below. In one embodiment, the risk score may be determined according to the formula:

$$Score = \sqrt{{\prod }_{x} {( H {R}_{x} )}^{x}} ∀ x ϵ [ tumour grade , tumour size ( cm ) , lymph node stage , age ( years ) , DI ]$$

Where:

Tumour grade, tumour size (in cm, for example), lymph node stage, age (in years, for example) and DI (which may also be referred to as the histological index) are types of data used as predictors/independent variables for the formula is based on.

$${\prod }_{x} ( … ) = the product of ( … ) for each variable .$$

$$H {R}_{x} = the hazard ratio for a particular variable .$$

x [where the hazard ratio is raised to the power of x]=the value (CFV) of the particular variable (e.g., tumour grade may equal 1, 2 or 3).

In other embodiments, a different formula may be used to determine the risk score. For example, a summation of values relating to different variables may be used instead of a product.

The values of each variable may be normalised. Various normalisation techniques are known in the art and any suitable normalisation technique may be used.

The hazard ratios may be determined as outputs of a logistic regression model, such as a survival regression model. One suitable example of a logistic regression model is the Cox regression model or proportional hazards regression model. Application of the Cox regression model involves fitting a model to data based on the time to occurrence of an event (such as death/death due to cancer/recurrence) since initial diagnosis. Coefficients, known as hazard ratios, are generated from fitting this model to a sample of suitable size.

The method 50 may comprise a further step (not shown) of using the prognosticated risk score to stratify the patient into one of at least two risk classifications with respect to the clinical outcome. For example, depending on the prognosticated risk score, the patient may be stratified as either ‘low-risk’ or ‘high-risk’. In other words, the patient is classified as being at low or high risk of experiencing the relevant clinical outcome with a predetermined time frame, such as five years or ten years. In some embodiments, stratifying the patient into one of at least two risk classifications may be done using a look-up table.

A ‘partial hazard’ may represent the relative change in risk of a particular event due to the value of the patient's variables and may be calculated as follows:

$$Partial Hazard = {\prod }_{x} {( H {R}_{x} )}^{x}$$

The method 50 may comprise a further step (not shown) of using the partial hazard to assign a likelihood of a particular event (such as death/death due to cancer/recurrence) occurring for a patient.

In some embodiments, assigning a percentage chance of a particular event may be done using a look-up table. In other embodiments, a survival probability may be calculated as follow:

$$Survival probability = exp ( - partial hazard \times cumulative baseline hazard )$$

The baseline hazard is the level of hazard experienced by a control group without any usage of a prognostic score.

The method 50 may be performed in an automated manner, for example, carried out entirely by the processor 12 shown in FIG. 1. Indeed, a computer program comprising computer code may be configured to perform the method 50. The processor 12 may be operated by such a computer program.

By virtue of this automation, the generation of a prognosticated risk score, or even stratification of the patient as low-risk or high-risk for example, can be carried out in a very short time period. From the time that sample analysis begins (e.g., once spectral analysis begins, after deparaffinisation) a result may feasibly be provided within just 15 minutes, or even as little as 5 minutes.

Furthermore, the apparatus 1 may be fabricated from components that are readily available, if not already being used in a variety of clinical settings for other purposes. Hence, the cost of goods and set-up required to carry out the invention are minimal such that the invention offers a particularly economic means for determining a prognosis of a patient while also avoiding the extended waiting time for test results and decisions regarding treatment that adds to a patient's anxiety and stress.

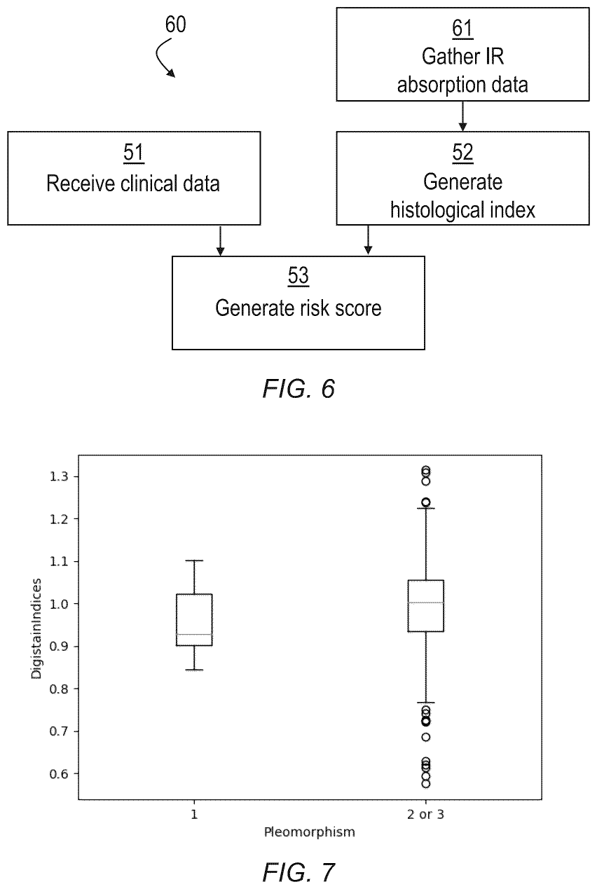

Referring now to FIG. 6, a method 60 is shown which includes the steps of the method 50 shown in FIG. 5 and further includes gathering the infrared absorption data from the sample (ex vivo) at selected wavelengths (step 61) is shown as an independent step separate to the generation of the histological index (step 52).

Step 61 may be carried out using the apparatus 1 shown in FIG. 1. The gathered infrared absorption data may be transferred to a separate processor configured to carry out steps 51 to 53 (i.e., the method 50 shown in FIG. 5).

Alternatively, the entire method 60 of FIG. 6 may be carried out by a single apparatus.

Such an apparatus for prognosticating risk of a clinical outcome based on a tissue sample of a patient may comprise a detector configured to obtain infrared absorption data from a tissue sample at selected wavelengths. The apparatus may also comprise a processor/processing module configured to process said infrared absorption data and receive clinical data related to the patient. The processing module may be configured to carry out the method 50 of FIG. 5 and therefore the apparatus, as a whole, would be configured to carry out the method 60 of FIG. 6.

In other words, the apparatus 1, shown in FIG. 1, may be suitable to carry out the method 60 of FIG. 6 provided that the processor 12 is configured to carry out steps 51 to 53.

In summary, by combining a histological index with selected clinical data using the methods, computer programs and apparatus described above, risk prognostication may be performed with comparable, and potentially improved, accuracy over known prognosticative methods while also being able to provide faster results. Accordingly, anxiety and stress which may be experienced by patients waiting for test results and decisions regarding treatment may be reduced.

The effectiveness of combining a histological index with selected clinical data is particularly surprising considering how cost-effectively the risk prognostication technique may be introduced into existing medical clinics and the like. The technique may be carried out using hardware that is readily available in the field and software that can easily be integrated into the necessary systems. Therefore, with minimal expenditure it may possible to implement risk prognostication that is more informative a histological index considered in isolation and able to provide faster results than known alternative methods.

### Patient Cohort and Methods

The methods, computer programs and apparatus described previously with reference to FIGS. 1 to 6 have been used in a number of trials to demonstrate their efficacy in prognosticating risk of clinical outcomes.

### Study Cohort, Clinicopathological Data, and Outcomes

Early stage, primary operable invasive breast cancer (BC) patients (N=801) aged ≤70 years, hormone positive (HR+), HER2 negative (HER2−) and with up to 3 positive lymph nodes (LN+≤3), were identified through multidisciplinary team records at Nottingham City Hospital, UK, where they were treated between 1998 and 2006. The median follow-up time for these patients was 12.7 years and the median age at diagnosis was 53 years. Hormone receptor status (ER and PR) and HER2 were assessed immunohistochemically using tissue microarray according to standard procedures. ER and PR positivity were defined as ≥1% nuclear staining.

Patient management was uniform, including systemic endocrine therapy where appropriate, and based on tumour characteristics by NPI and hormone receptor status. Patients within the NPI excellent prognostic group (score ≤3.4) received no adjuvant therapy, but those patients with NPI >3.4 received tamoxifen if HR-positive [±Goserelin (Zoladex) in premenopausal patients]. None of the patients in this series received neoadjuvant therapy. In this cohort, clinical history, tumour characteristics and outcome data are prospectively maintained and regularly updated from electronic patient records and pathological/histopathological reports. Patients were excluded if, no tumour was present, if the tumour type was identified as ductal carcinoma in situ (DCIS), if they were treated with chemotherapy or neoadjuvant therapy, if the oestrogen receptor status was unknown or negative and finally if no long-term follow up was available.

Clinical history and tumour data collected include patient's age at diagnosis, menopausal status, tumour size, tumour grade, tumour type, NPI, nodal stage and lymphovascular invasion. The outcome data recorded include 5-year and 10-year Overall Survival (OS), Disease Free Survival (DFS) and Recurrence, defined as the time, in months, from the date of primary surgery to the respective event.

### Histological Indexing Procedure

Clinical tissues samples were de-identified and two adjacent sections, each ˜4 μm thick, were cut from each tissue microarray (TMA) block; one section was hematoxylin and eosin (H&E) stained and graded according to the Elston-Ellis method for NPI scoring. The second serial section was mounted on an IR-transmitting calcium fluoride microscope slide and after deparaffinization was placed in a Bruker Vertex70 Infrared Spectrometer equipped with a Hyperion 2000 microscope, an example of the apparatus shown in FIG. 1. The microscope aperture was set to sample an area of 500 microns×500 microns (smaller than the area of the core). The aperture was centred over each core and an average of 64 interferograms was then recorded for each unstained core section on the slide. The resulting averaged interferogram for each sample was Fourier Transformed and thus converted to an absorption spectrum using Bruker's OPUS software. A histological index (or Digistain Index, DI) was quantified using proprietary software written in MATLAB version R2022b.

### Statistical Analysis

The DI was summarized descriptively using standard descriptive statistics (mean, median, standard deviation, minimum and maximum value). Data transformations, such as logs or inverse, were considered for non-normal distributions and the distribution of the transformed variables was summarized.

Kaplan-Meier curves were used to display the overall, disease-fee survival time and recurrence for high and low risk patients. Median and 95% confidence intervals are reported where they can be calculated. The length of follow up is reported descriptively.

Modelling methods followed the approach recommended by Royston et al., A multivariate logistic model modelling the ‘event’ (death, disease-specific death or recurrence) by 5-year or 10-year was used. The model assumptions included:

1) Due to relatively small number of events the sample was not split into training and validation, instead bootstrapping (n=500) was used to assess performance.

2) Five candidate predictors were included. Those known to be prognostic (age, tumour grade and size, and lymph node status) plus the novel marker of interest (DI).

3) No model selection took place.

4) Age has an approximately normal distribution and was included as a continuous predictor (odds ratios calculated based on a 10-year increase in age); Tumour grade (Grade 1, 2, 3) and lymph node status (“1” for negative and “2” for 1-3 positive lymph nodes, were modelled as a categorical variable with reference/dummy coding using grade/status 1 as the reference category.

5) Tumour size was modelled as continuous predictor. Natural logs were used due to skewness. Odds ratios were calculated based on an increase of 0.7 on the logged value (i.e., an approx. increase of 2 mm).

6) Calculation of beta coefficients for the risk prediction formula from “Bootstrapping Introduction to Bootstrapping Simulation in SAS”.

(https://documentation.sas.com/doc/en/pgmsascdc/9.4_3.4/statug/statug_ttest_details20.htm).

The relationship between clinical categorical and/or continuous variables and 5-year or 10-year clinical outcomes was analysed using Cox proportional hazards regression models. The variables considered included the DI, tumour size, grade, age at diagnosis, NPI, lymph node status, HR status and Her2 status. A Receiver Operator Characteristics (ROC) curve was constructed and the Area Under the ROC Curve (AUC) calculated, with an AUC of 1 representing perfect prediction and 0.5 representing random prediction (i.e., a test of no value).

### Results

### Study Cohort, Clinicopathological Data, and Outcomes

The retrospective analysis included data from 801 patients who were HR+, HER2− and had 0-3 positive lymph nodes, with follow-up data for 5 and 10 years. The mean age of the patients at diagnosis was 53.9 years (median=54.5 years). A summary of patient tumour tissue characteristics is shown in Table 3. All 801 patients were HR+ and HER2− and among them 584 (68.41%) were lymph node negative and 253 (31.59%) had 1 to 3 positive lymph nodes. The majority of patients, 85.8% (n=681), had invasive ductal carcinoma and 10.36% (n=83) had invasive lobular cancer, while the remaining were either mixed, special type or tubular. In terms of tumour grade, 20.97% (n=168), 49.31% (n=395) and 29.71% (n=238) were of histological grade 1, 2 and 3, respectively. At the time of diagnosis, the risk classification based on NPI was as follows: 46.32% (n=371), 46.32% (n=371) and 7.24% (n=58) with NPI score of good (>2.4 but ≤3.4), moderate (>3.4 but ≤5.4) and poor (>5.4), respectively (with data for one patient missing).

TABLE 3
Summary of tumour tissue characteristics (N =
801; all HR+ and HER2−).

| | |
|---|---|
| N= | % |

Tumour Type

| | | |
|---|---|---|
| Ductal (including | 681 | 85.02 |
| Lobular | 83 | 10.36 |
| Mixed NST and Lobular | 1 | 0.12 |
| Special type | 35 | 4.37 |
| Tubular | 1 | 0.12 |

Tumour Grade

| | | |
|---|---|---|
| 1 | 168 | 20.97 |
| 2 | 395 | 49.31 |
| 3 | 238 | 29.71 |

Lymph node status

| | | |
|---|---|---|
| 1 (negative) | 548 | 68.41 |
| 2 (1-3 positive) | 253 | 31.59 |

Pleomorphism

| | | |
|---|---|---|
| 1 | 17 | 2.12 |
| 2 | 300 | 37.45 |
| 3 | 477 | 59.55 |
| Missing data | 7 | 0.87 |

NPI

| | | |
|---|---|---|
| GPG | 371 | 46.32 |
| MPG | 371 | 46.32 |
| PPG | 58 | 7.24 |
| Missing data | 1 | 0.12 |

Recurrence

| | | |
|---|---|---|
| No | 642 | 80.15 |
| Yes | 159 | 19.85 |

(GPS: Good Prognostic Score; HER2: Human Epidermal Growth Factor Receptor 2; HR: Hormone Receptor; MPG: Moderate Prognostic Score; NPI: Nottingham Prognostic Index; PPG: Poor Prognostic Score.)

The mean length of follow up (from patient diagnosis to last follow up) was 12 years. The median was also 12 years (0.9 to 19 years). A summary of survival data after 5 and 10 years is shown in Table 4 and Table 5, respectively.

TABLE 4
Summary of 5-year survival status.

| 5-year survival | N = 801 | % |
|---|---|---|
| Alive at 5 years, died later | 129 | 16.10 |
| Alive at more than 5 years | 601 | 75.03 |
| Died before 5 years | 44 | 5.49 |
| Follow up less than 5 | 27 | 3.37 |

TABLE 5
Summary of 10-year survival status.

| 10-year survival | N = 801 | % |
|---|---|---|
| Alive at 10 years, died | 59 | 7.37 |
| Alive at more than 10 | 472 | 58.93 |
| Died before 10 years | 114 | 14.23 |
| Follow up less than 10 | 156 | 19.48 |

### Histological Index (DI) Description

### DI Distribution and Cut-Offs

The mean DI value was 0.9 (SD 0.09) with a minimum of 0.58 and a maximum of 1.31 (Table). The distribution curve indicated that DI values was more skewed than what would be expected from a variable showing normal distribution, and this remained the case even when log or inverse transformation was applied. Nevertheless, considering that based on mean and median DI did not deviate much, it was agreed to proceed with further analyses using DI as a normally distributed variable.

DI cut-offs were selected as those values that generate the lowest significance value (p) in the Kaplan Meier curves.

TABLE 6
Histological index (DI) values distribution.

| | Mean | SD | Median | Min | Max | N | N Missing |
|---|---|---|---|---|---|---|---|
| DI | 0.99 | 0.09 | 1.00 | 0.58 | 1.31 | 797 | 4 |

The inventors sought to examine the link between the biological concept underpinning the histological index and clinical prognosis. Considering that DNA aneuploidy has been shown to correlate with a high malignancy grade, frequent mitoses, a high degree of nuclear pleomorphism and the difficulties in assessing aneuploidy in tissue sections, the inventors used pleomorphism as a surrogate for aneuploidy and examined its relationship to the histological index (or Digistain Index, DI). The distribution of DI for each of the three pleomorphism scores is shown in FIG. 7.

### Accuracy of Histological Index-Based Risk Prognostication

Overall, AUC values for the ROC curve obtained from the 5-year analysis for all three clinical outcomes examined (death, disease-specific death, and recurrence) and across all patient groups was consistently high. The AUC values for the ROC curves obtained at the 10-year analysis were lower than those at 5-years but still remained within good predictive levels across all clinical outcomes (data summarized in Table 8 and in Table 9).

In the following analysis, the outcomes ‘disease specific death’ and ‘disease free survival’ may be used interchangeably on the basis that a positive test result would indicate disease specific death and a negative test result would indicate disease free survival. Similarly, the outcomes ‘death’ and ‘overall survival’ may be used interchangeably for the same reason.

The outcomes ‘disease free survival’ and ‘progress free survival’ may also be used interchangeably in the context of this disclosure on the basis that both outcomes are considered as being equivalent in a medical/clinical setting.

TABLE 8
Accuracy (AUC under ROC curve), PPV and NPV of DI-based
risk scoring for 5-year clinical outcomes (N = 801).

| | | | | HR+/HER2−/ | HR+/HER2−/ |
|---|---|---|---|---|---|
| | | HR+/ | HR+/ | LN+ ≤ 3 & | LN+ ≤ 3 & |
| 5-year | | HER2−/ | HER2−/ | age ≤ 45 years | age ≥ 60 years |
| analysis | | LN+ ≤ 3 | LN− | (pre-M) | (post-M) |
| NPV | DFS | 0.99 | 0.99 | 0.98 | 0.98 |
| | Rec. | 0.99 | 0.99 | 0.98 | 0.98 |
| | OS | 0.97 | 0.97 | 0.98 | 0.96 |
| PPV | DFS | 0.06 | 0.02 | 0.07 | 0.05 |
| | Rec. | 0.07 | 0.02 | 0.07 | 0.06 |
| | OS | 0.08 | 0.03 | 0.08 | 0.08 |
| AUC | DFS | 0.81 | 0.71 | 0.73 | 0.71 |
| | Rec. | 0.81 | 0.71 | 0.73 | 0.72 |
| | OS | 0.77 | 0.67 | 0.76 | 0.70 |

(AUC: Area Under the Curve; CI: Confidence Intervals; DFS: Disease Free Survival; HR: Hazard Ratio; HER2−: Her2 negative; HR+: Hormone Receptor positive; LN−: lymph node negative; LN+ ≤ 3: with up to 3 positive lymph nodes; NE: not estimable; OS: Overall Survival; ROC: Receiver Operating Characteristics curve.)

TABLE 9
Accuracy (AUC under ROC curve), PPV and NPP of DI-based
risk scoring for 10-year clinical outcomes (N = 801).

| | | | | HR+/HER2−/ | HR+/HER2−/ |
|---|---|---|---|---|---|
| | | HR+/ | HR+/ | LN+ ≤ 3 & | LN+ ≤ 3 & |
| 10-year | | HER2−/ | HER2−/ | age ≤ 45 years | age ≥ 60 years |
| analysis | | LN+ ≤ 3 | LN− | (pre-M) | (post-M) |
| NPV | DFS | 0.94 | 0.95 | 0.93 | 0.92 |
| | Rec. | 0.94 | 0.95 | 0.93 | 0.92 |
| | OS | 0.90 | 0.90 | 0.91 | 0.84 |
| PPV | DFS | 0.15 | 0.10 | 0.17 | 0.14 |
| | Rec. | 0.15 | 0.11 | 0.16 | 0.14 |
| | OS | 0.18 | 0.12 | 0.18 | 0.21 |
| AUC | DFS | 0.75 | 0.70 | 0.62 | 0.74 |
| | Rec. | 0.75 | 0.69 | 0.63 | 0.72 |
| | OS | 0.69 | 0.62 | 0.59 | 0.66 |

(AUC: Area Under the Curve; CI: Confidence Intervals; DFS: Disease Free Survival; HR: Hazard Ratio: HER2−: Her2 negative HR+: Hormone Receptor positive; LN−: lymph node negative; LN+ ≤ 3: with up to 3 positive lymph nodes; OS: Overall Survival; ROC: Receiver Operating Characteristics curve.)

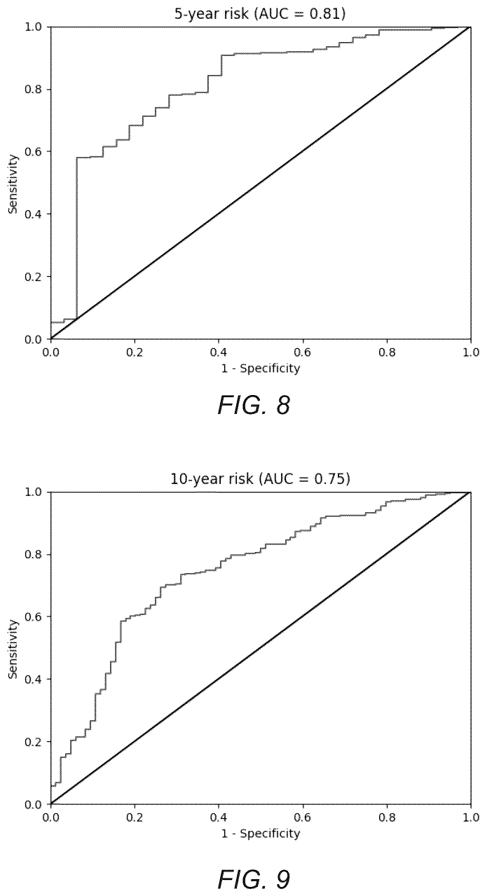

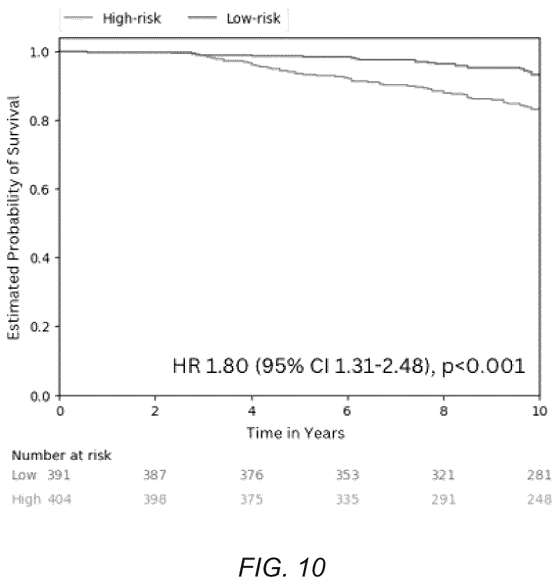

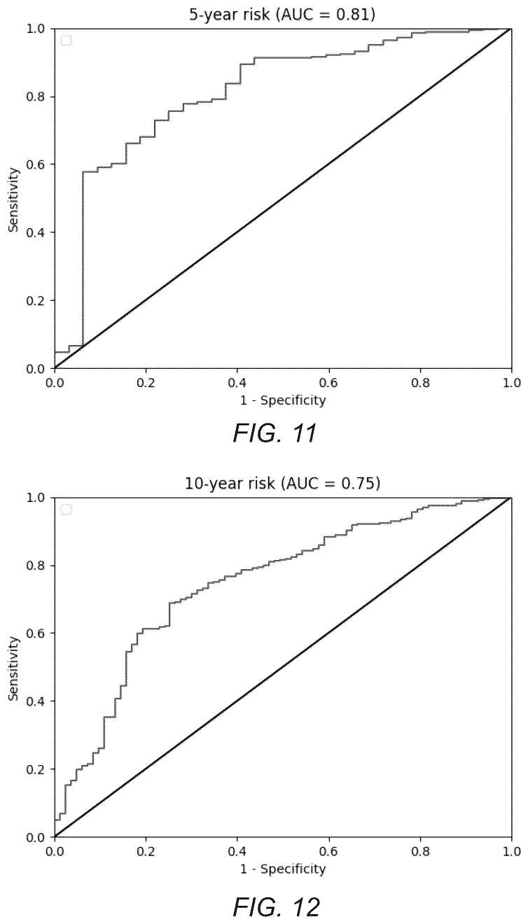

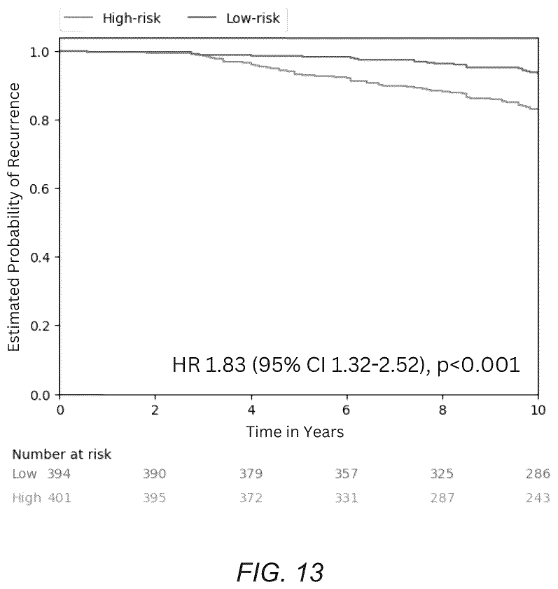

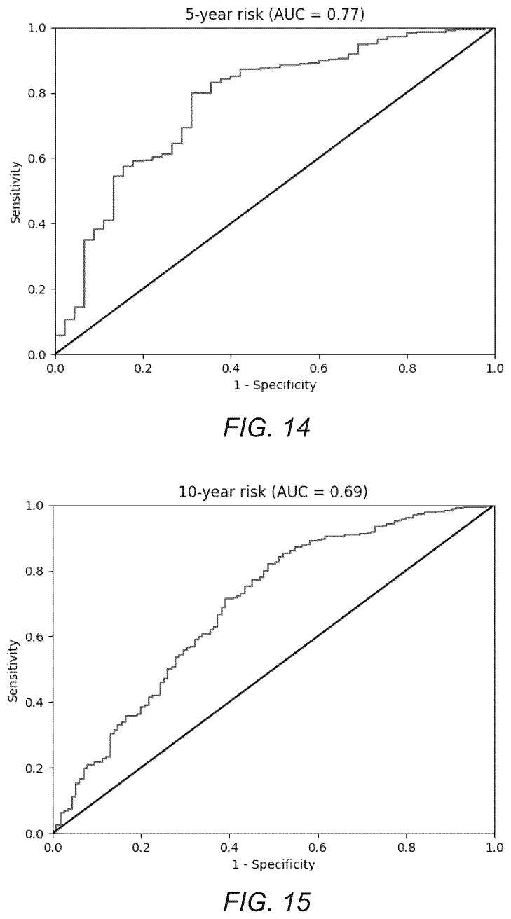

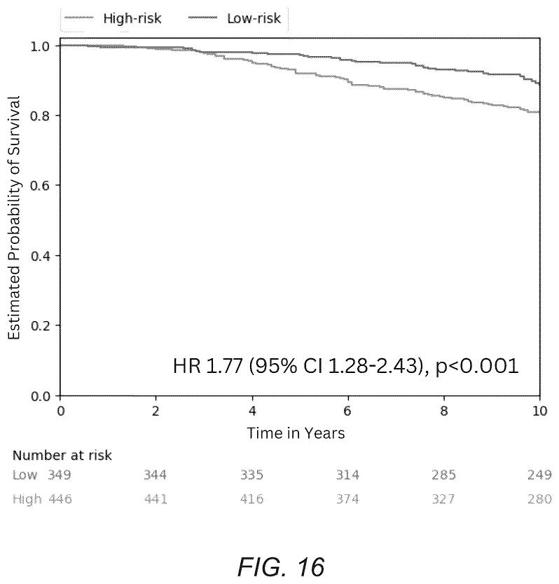

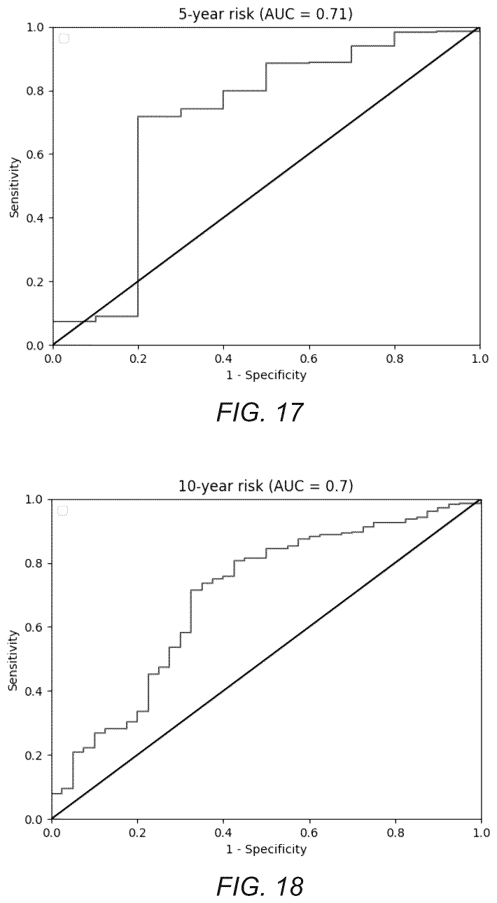

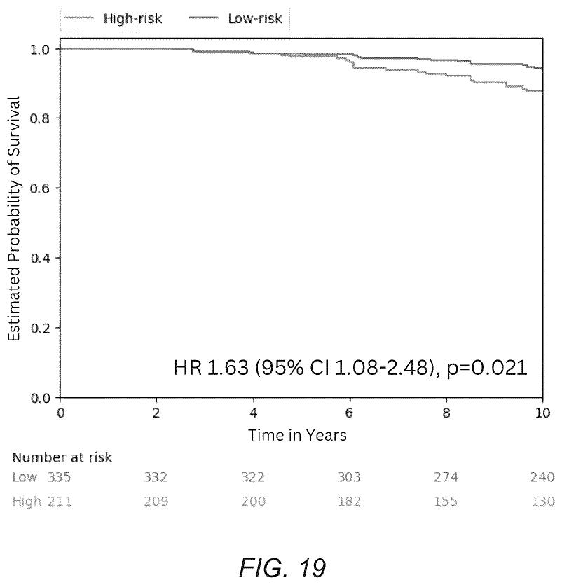

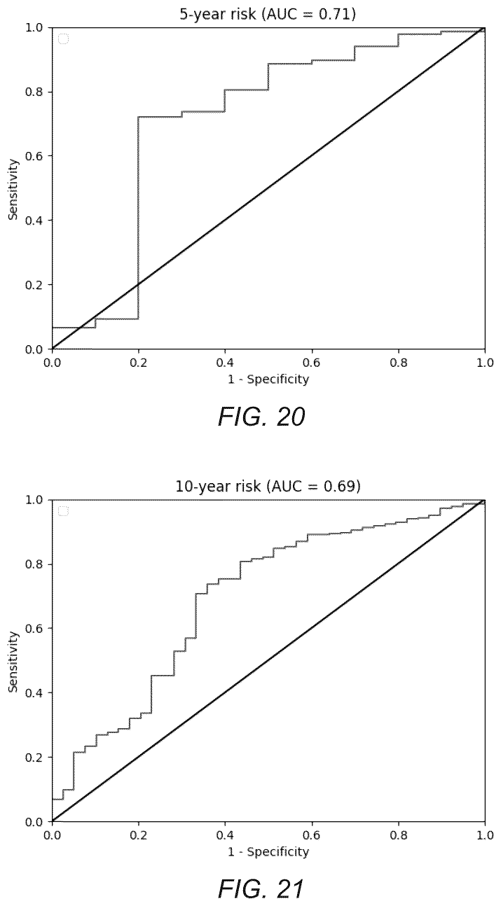

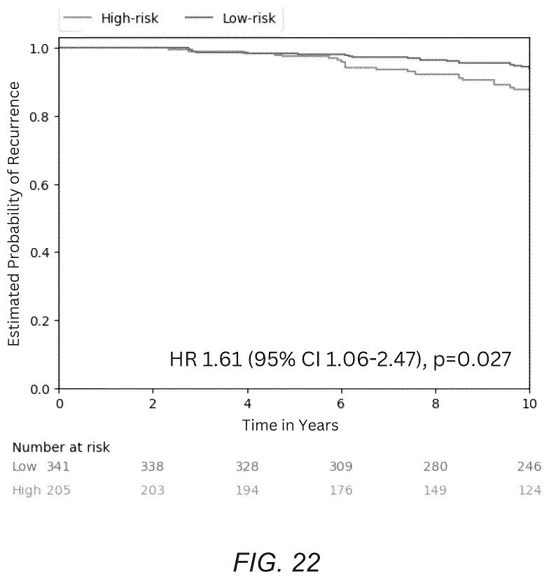

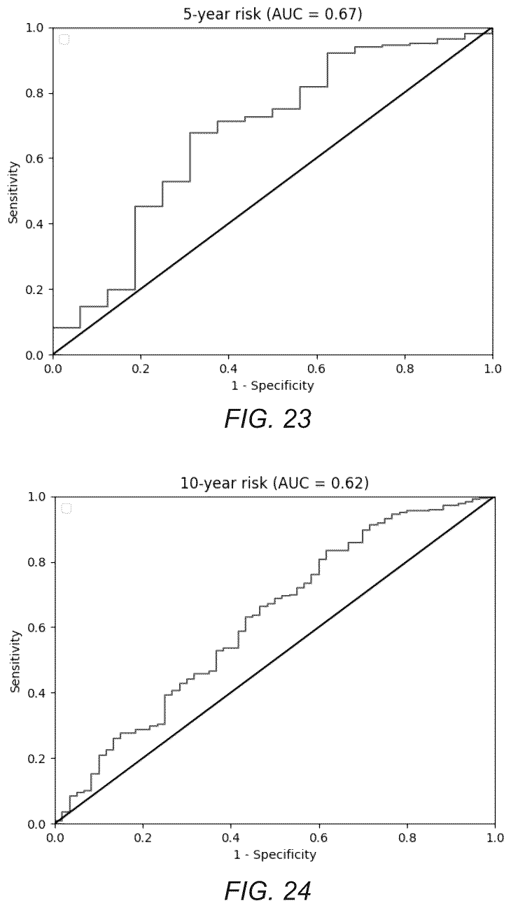

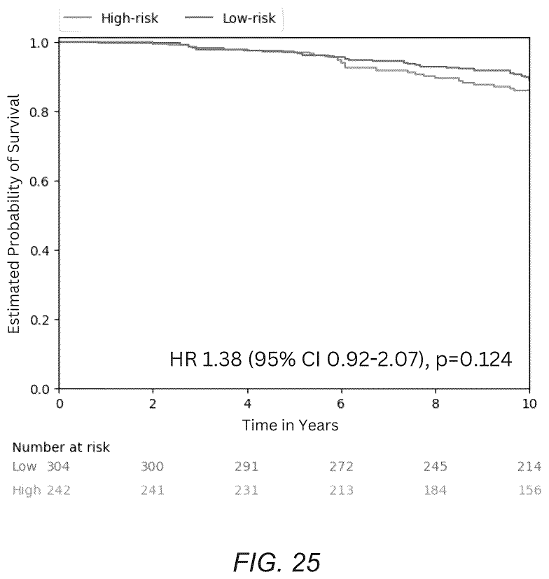

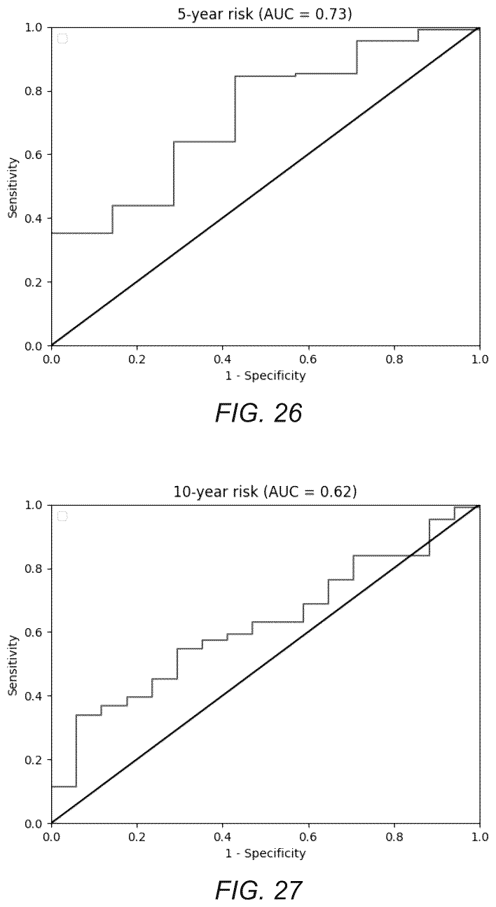

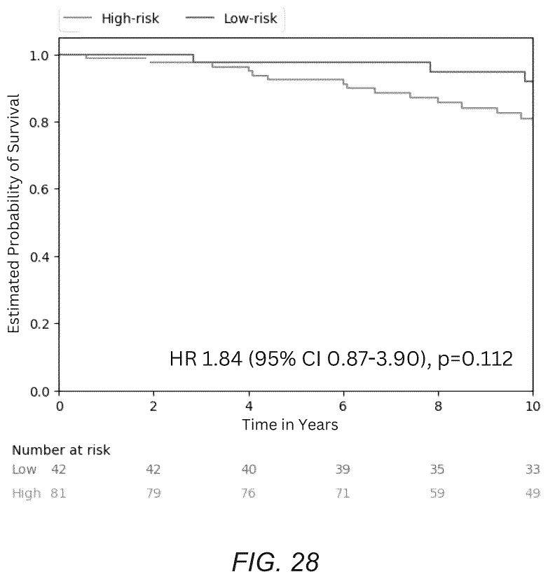

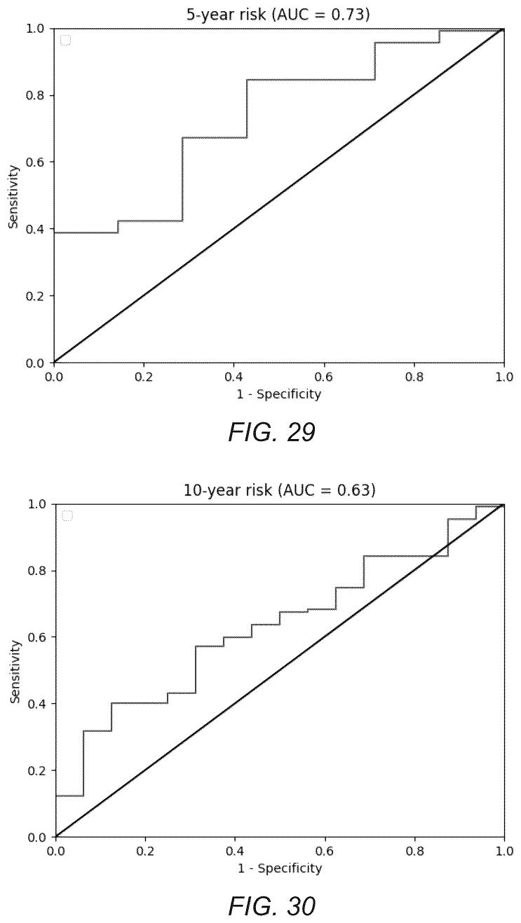

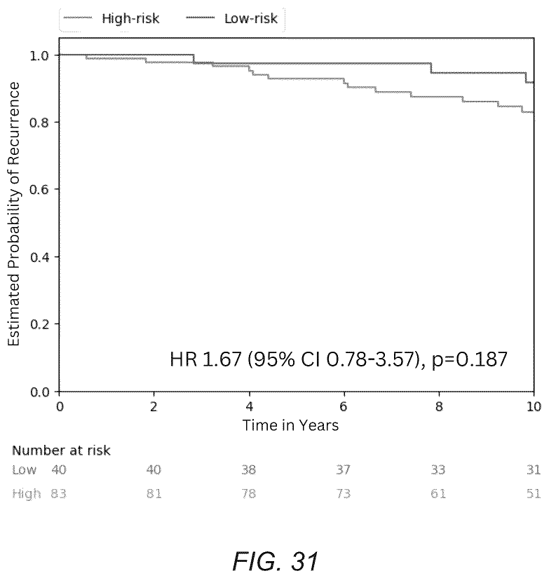

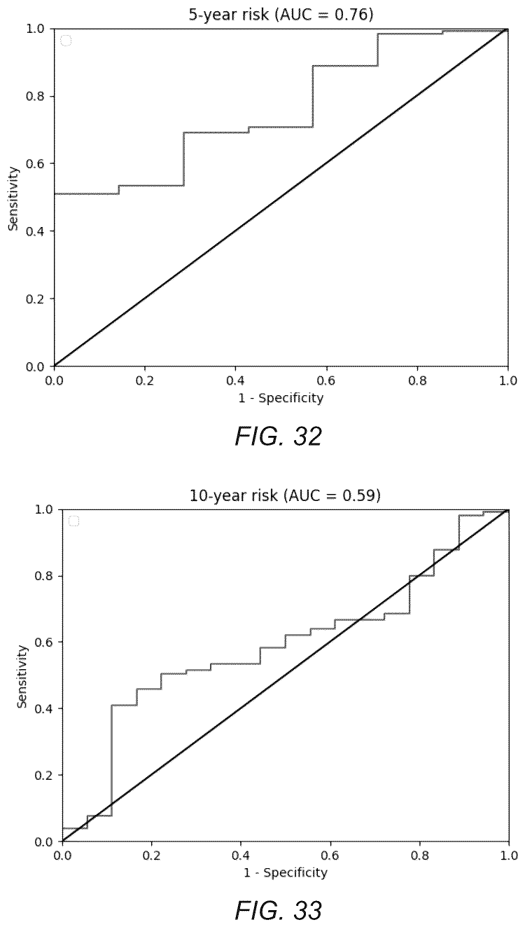

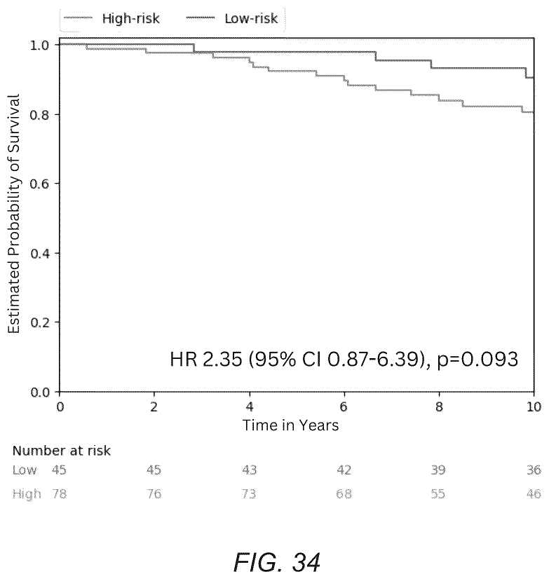

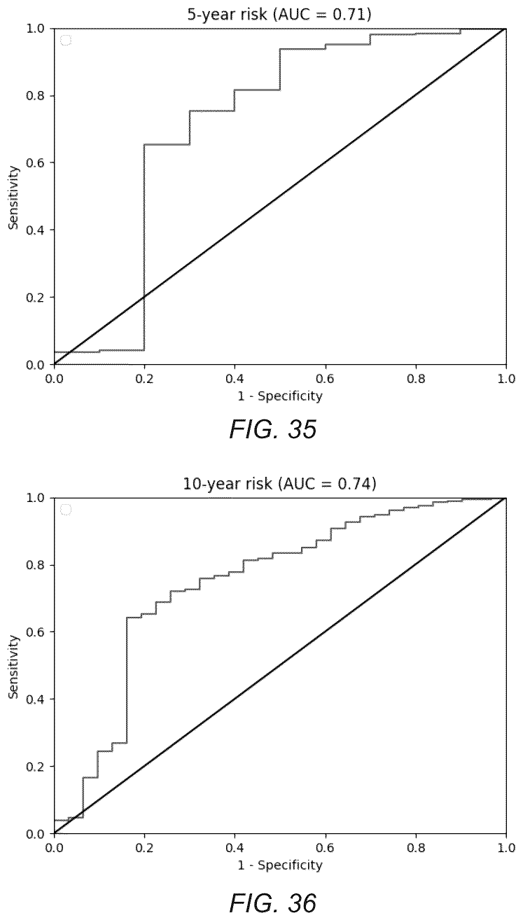

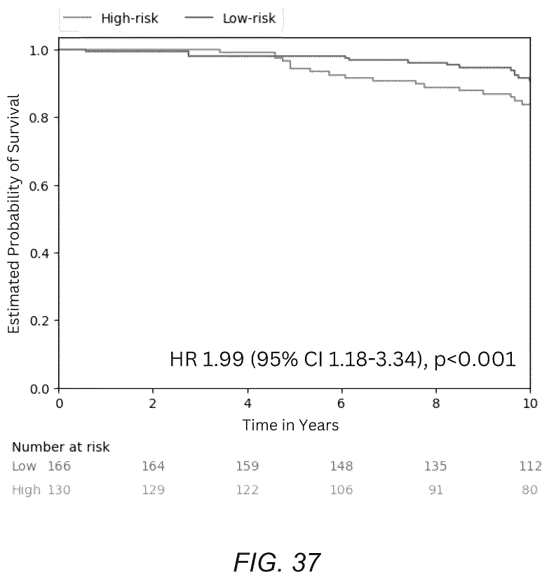

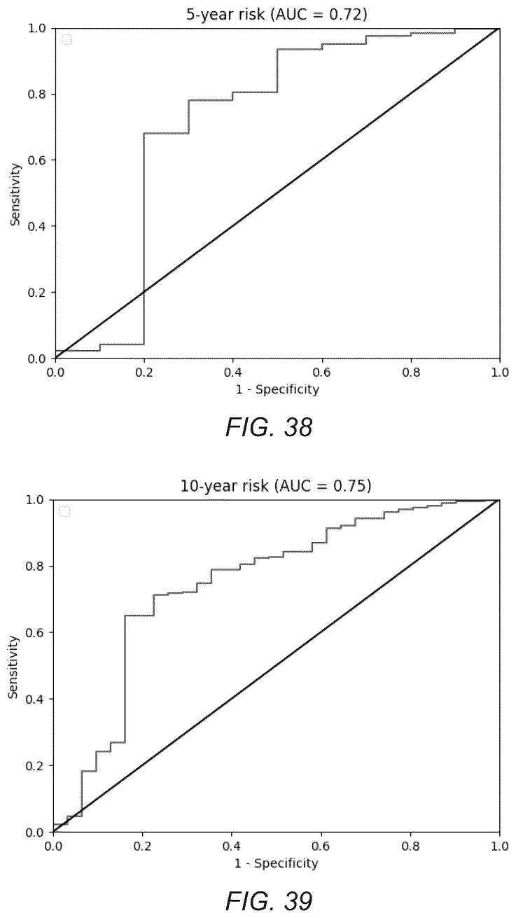

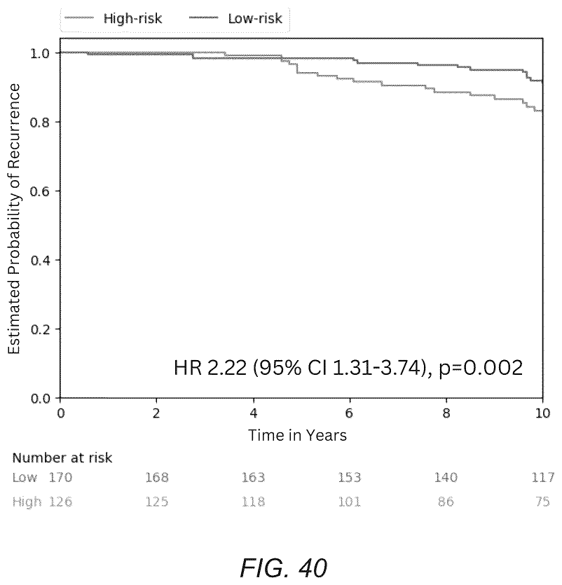

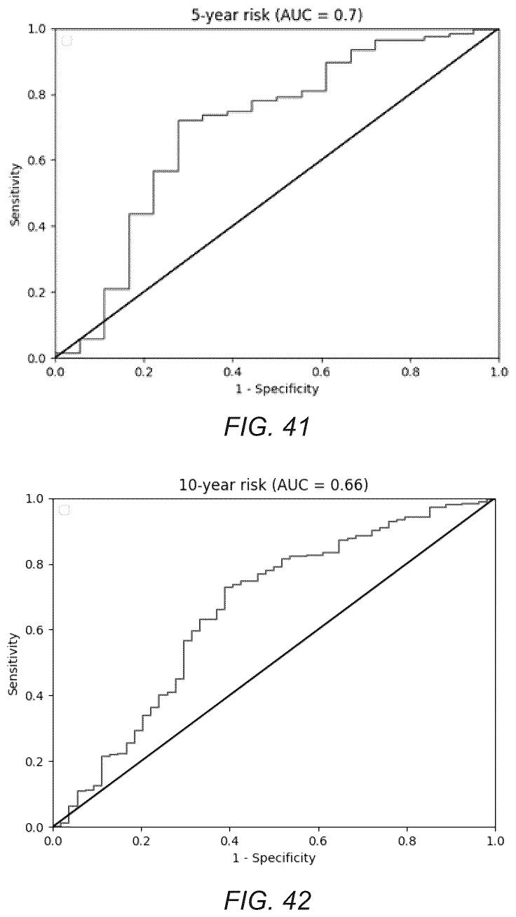

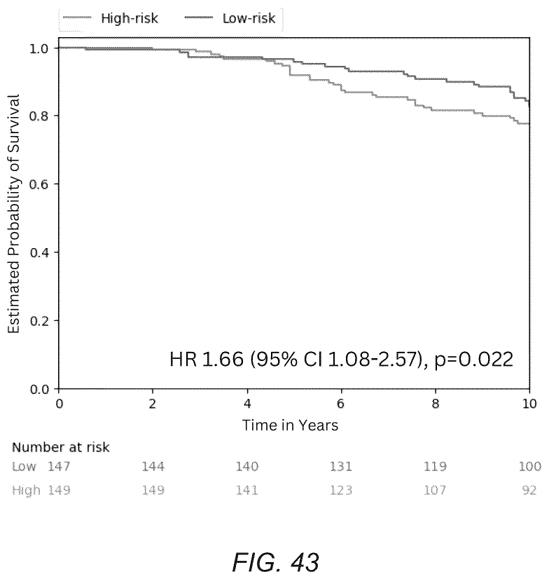

Among the four groups examined, the AUC values were the highest in the HR+/HER2−/LN+≤3 group for all of disease free survival, recurrence and overall survival. Values were the same for disease free survival and recurrence, with 0.81 and 0.75 at 5-year and 10-years, respectively. For overall survival the AUC values were 0.77 and 0.69 at 5-year and 10-years, respectively. (See FIGS. 8, 9, 11, 12, 14 and 15.)

In particular, FIGS. 8 and 9 show graphs illustrating histological index accuracy (AUC under ROC curves) at 5-years and 10-years, respectively, for predicting disease free survival in HR+/HER2−/LN+≤3 patients. Further detail on the results for this group of patients is provided in Table 10 below.

From the foregoing, it will be appreciated that specific embodiments of the invention have been described herein for purposes of illustration, but that various modifications may be made without deviating from the scope of the invention. Accordingly, the invention is not limited except as by the appended claims.

I claim:

1. An automated method for prognosticating risk of a clinical outcome for a patient, the method comprising:
receiving clinical data relating to the patient;
generating a histological index based on infrared absorption data gathered from a sample of the patient; and
generating a prognosticated risk score based on the histological index and the clinical data.

2. The method of claim 1, wherein generating the prognosticated risk score comprises incorporating the histological index and the clinical data in a model.

3. The method of claim 2, comprises determining a hazard ratio calculated using a logistic regression model for each of the histological index and the clinical data.

4. The method of claim 1, further comprising:
using the prognosticated risk score to stratify the patient into one of at least two risk classifications with respect to the clinical outcome.

5. The method of claim 1, further comprising:
determining one or more clinicopathological factor values based on the clinical data,
wherein the or each clinicopathological factor value is used to generate the prognosticated risk score.

6. The method of claim 1, wherein the clinical outcome comprises one or more from a list comprising:
death;
disease-specific death;
recurrence; or
complete pathological response.

7. The method of claim 1, wherein the patient is a patient previously diagnosed with a cancer, wherein the clinical data is an attribute of the patient associated with prognosis for the previously diagnosed cancer.

8. The method of claim 1, wherein the clinical data comprise one or more from a list comprising:
age when diagnosed with cancer;
menopausal status;
tumour size;
tumour grade; and
lymph node status.

9. The method of claim 1, wherein generating the histological index based on infrared absorption data gathered from the sample comprises:
gathering infrared absorption data from the sample at selected wavelengths;
determining, from the infrared absorption data, a first measure of an amount of energy or power absorbed attributable to an amide moiety and a second measure of the amount of energy or power absorbed attributable to a phosphate moiety; and
determining a ratio of the first measure and the second measure to establish the histological index.

10. The method of claim 9, wherein the histological index comprises a numeric value obtained by dividing the first measure by the second measure.

11. The method of claim 9, wherein the histological index, PA, is derived according to expression PA=[|M(λ3)−M(λ4)|]/[|M(λ1)−M(λ2)|] where: M(Δn) is a measure of the absorbed energy or power at Δn; Δ1 is a wavelength corresponding to a peak absorption value attributable to an amide moiety; λ2 is a wavelength corresponding to a baseline absorption value attributable to an amide moiety; Δ3 is a wavelength corresponding to a peak absorption value attributable to a phosphate moiety; λ4 is a wavelength corresponding to a baseline absorption value attributable to a phosphate moiety.

12. The method of claim 9, wherein the histological index, PA, is derived according to expression PA=[X3 M(λ3)−X4 M(λ4)]/[X1 M(λ1)−X2 M(λ2)] where: M(λn) is a measure of the absorbed energy or power at λn; λ1 is a wavelength corresponding to a peak absorption value attributable to an amide moiety; λ2 is a wavelength corresponding to a baseline absorption value attributable to an amide moiety; λ3 is a wavelength corresponding to a peak absorption value attributable to a phosphate moiety; λ4 is a wavelength corresponding to a baseline absorption value attributable to a phosphate moiety; and X1 to X4 are numerical factors ≥1 which are set to values sufficient to ensure that measure M for a peak absorption values λ3 and λ1 is always greater than the measure M for the corresponding baseline absorption values λ4 and λ2 for all measurements.

13. The method of claim 1, wherein:
the method further comprises gathering infrared absorption data from the sample at selected wavelengths; and
generating a histological index based on infrared absorption data gathered from a sample of the patient comprises generating a histological index based on the gathered infrared absorption data.

14. The method of claim 13, wherein the infrared absorption data are gathered using an interferometer, Raman spectroscopy spectral imager, spectral detector and/or a wavelength-tuneable light source.

15. The method of claim 13, wherein the sample is a tissue sample.

16. The method of claim 13, wherein the sample is from 1 μm to 10 μm thick, preferably about 4 μm thick.

17. The method of claim 13, wherein the obtained infrared absorption data relates to a single spatial position on the sample.

18. An apparatus for prognosticating risk of a clinical outcome based on a sample of a patient, comprising:
a detector configured to obtain infrared absorption data from a tissue at selected wavelengths; and
a processing module configured to process said infrared absorption data and receive clinical data related to the patient,
wherein the apparatus is configured to carry out the method of any one of claim 14.

19. The apparatus of claim 18, wherein the detector is comprised by an interferometer.

20. A computer program comprising computer code configured to cause one or more processors to perform the method of claim 1.
